(19) United States
(12) Patent Application Publication
(10) Pub. No.: US 2008/0104727 A1
(43) Pub. Date: May 1, 2008

(54) NAMING HYBRIDIZATION BUSINESS

(76) Inventor: Robert Hunt, Boise, ID (US)

Correspondence Address: DYKAS, SHAVER & NIPPER, LLP, P.O. BOX 877, BOISE, ID, 83701-0877, US

(21) Appl. No.: 11/927,505

(22) Filed: Oct. 29, 2007

Related U.S. Application Data
(60) Provisional application No. 60/863,145, filed on Oct. 27, 2006.

(51) Int. Cl. G06Q 30/00; A01H 1/00

(52) U.S. Cl. 800/278; 705/1

(57) ABSTRACT

The invention is a business method of producing genetically unique plants such as Gladiolus, with each unique variety including bulbs for propagation, and offering a customer the opportunity to name the new variety of plant in memory of or in honor of a person of his choice.

### CROSS-REFERENCE TO RELATED APPLICATIONS

This application claims the priority date of the provisional application entitled NAMING HYBRIDIZATION BUSINESS filed by Robert Hunt on Oct. 27, 2006 with application Ser. No. 60/863,145.

### FIELD OF THE INVENTION

The invention relates generally to a system for naming unique species of plants for customers, and more particularly to producing genetically unique Gladiolus plants, and naming the unique species for a customer of the service.

### BACKGROUND OF THE INVENTION

There is a desire among many people to have a product which either memorializes or honors a person. Examples of this are in registries that purport to name a star after a person. Very few other mechanisms exist for honoring or memorializing a person. Of much greater utility would be a memorial which could be shared with family members, which could be a living thing of beauty and interest, and which could be shown to be truly registered for the person intended. At present no such system exists except that of the invention.

As long as there are people in love, there will be stress, anxiety, and even desperation associated with gift giving. Gift giving is HUGE and getting bigger all the time. Barring the extinction of relationships as a whole, we expect this trend to continue. And the increase is not merely in the volume and frequency of gifts given. Several factors in today's world have seriously upped the ante on gift giving and receiving expectations.

The internet has expanded the proverbial shopping mall, bringing specialty items from around the world directly to our doorsteps. You can even buy super premium ice cream from small creameries across the country and have it delivered—still frozen—to your table. This means that the options for the gift giver have expanded exponentially, as have the expectations of the potential gift recipient.

The desire for personalization has become pervasive throughout many aspects of society. People choose, and even create custom ring tones for their cell phones, even for individual callers. Custom play lists are the norm for music listeners.

Technology has combined with the desire for personalization to enable “mass customization,” or the ability to provide truly customized products at the mass level. The importance of this movement is evidenced by the fact that major worldwide brands are making huge investments in the field. One of the best examples of mass customization is NikeID.com, where sneaker lovers can customize the appearance of numerous models of shoes, choosing distinct colors for soles, airbags, laces, eyelets, backgrounds, accents, the famous “swoosh” and can even add their initials or a signature line in the color of their choice.

Advances in e-commerce capabilities have been a key enabler of the mass customization process. The availability of low cost server hardware combined with open source database software and widespread broadband adoption has allowed merchants to create extensive and dynamic online storefronts. Conducting business online allows retailers to bypass many traditional barriers to market entry such as catalog printing (and distribution) costs and retail space rental. The global reach of the internet allows small niche products/services that might never have sufficient market in any geographical area under traditional brick-and-mortar business models to be quite successful online.

The possibilities are endless when choosing a gift for a loved one. Expectations of both gift givers and receivers are evolving based on their ability to personalize other aspects of their lives. A gift is sought which expresses the depth of emotion in a relationship, and which can capture the uniqueness of a human being. One which can truly acknowledge, honor, and even immortalize a loved one would be highly desirable. This is achieved by a unique variety of flower, never before available, named after a person.

### SUMMARY OF THE INVENTION

The present invention is a business method which includes the following steps. The first step is obtaining a plurality of genetically distinct living plants. One type of plant which lends itself to this step is the Gladiolus plant. When a variety of Gladiolus is crossed with another Gladiolus, the number of possible combinations is so large that it is statistically provable that no two crosses can be the same genetically. The next step is to create a display of the different individual plants that are available, such as pictures on a website or pictures in a catalog. From the pictures that are on the display of the available genetically distinct plants, the customer chooses one or more plants and purchases one or more plants. Along with the purchase of the living specimens of the plant, the customer also buys the right to name the plant. Thus, a plant could be named for a person that the customer knows, such as his wife, sweetheart, or mother. After the customer has selected a plant for purchase, and has chosen a name for that plant, the name is assigned to that variety of plant and the plant is then removed from the display of available names.

In addition to buying the right to name a plant, the customer can receive one or more living specimens of the plants, such as in the form of bulbs. Thus, after purchasing the right to name the plant, the customer would be able to assign a name to that plant and receive a number of bulbs of that particular genetic makeup of plant. These bulbs could be shared with friends and family, or be planted in memory of a person, or given to a person to honor and memorialize an event. This business method thus provides a unique and previously unavailable product that will help people express their love and affection for special people in their lives. It provides a distinctive, memorable, and lasting gift that will honor their love for the recipient for years to come and may be shared with friends and loved ones.

In addition to receiving bulbs of the selected plant, the customer may receive a framed photo of the flower in bloom and a pedigree certificate documenting the flowers parentage, along with a number of flower bulbs for planting.

The system of the invention allows the customer to honor the special people in their lives in a unique way. They have the privilege of immortalizing their loved ones with a beautiful flower named after them. This may all take place on the website, where users will be able to select their new, unnamed variety of flower based on color, flower shape, appearance, or even parentage/pedigree. They will then designate the name for the flower. The name is then documented through a special international registry. The customer receives a high-end package containing 5 to 10 actual bulbs so they can grow and share their flower, or present it to the person they would like to honor. In addition the customer may receive a framed certificate picturing their flower in bloom and a frameable pedigree certificate listing and usually picturing “parent flowers” that were crossed to create the new hybrid, now named after a particular person. Several lines for a personal dedication are also available.

Achieving genetically distinct plants is achieved by use of a plant called a Gladiolus. Gladioli are popular in the floral industry due to their strong presence and “showiness,” as well as the large variety of colors available. Gladioli have 60 chromosomes, which allows limitless variability. In comparison, Petunias have 8 chromosomes. When two varieties of Gladiolus are crossed, the result is a seed pod containing 20-100 seeds, all genetically distinct. Through a series of plantings, an appropriate number of saleable bulbs result. Given the thousands of existing Gladiolus varieties, all having 60 chromosomes each, the number of possible varieties is endless. Gladiolus is used as an example, but other plants may be used in the business method of the invention.

A package offered to the customer may include such items as a book on growing Gladiolus, membership to a grower's forum, and a membership to a botanical society.

Gladiolus bulbs are typically planted in the spring. When the plants grow and begin to bloom, the crossing of varieties, or hybridization, can begin. Normally, some Gladioli self pollinate. However, they can be cross-pollinated by placing the pollen from one variety onto the stigma of another. The seeds that then form contain a combination of genetic material from the two “parent” plants. Gladioli have a high number of chromosomes and produce a large number of seeds after pollination. Each of these seeds can be germinated to create a genetically distinct, new variety of Gladiolus. At the end of growing season after the seeds are first planted, the plant will have developed a “bulb,” which is then planted in the subsequent seasons and produces genetically identical “bulblets” which, when planted, grow, flower, and produce additional bulbs and bulblets. Each bulblet is genetically identical to the parent plant, since they are produced vegetatively. Starting from “parent bulbs” in the spring, it would take 4-5 growing seasons to produce enough “child bulbs” for a group of plants to be offered for sale, with pictures of their appearance when blooming.

The purpose of the foregoing Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other features and advantages of the present invention will become readily apparent to those skilled in this art from the following detailed description describing preferred embodiments of the invention, simply by way of illustration of the best mode contemplated by carrying out my invention. As will be realized, the invention is capable of modification in various obvious respects, all without departing from the invention. Accordingly, the drawings and description of the preferred embodiments are to be regarded as illustrative in nature, and not as restrictive in nature.

### BRIEF DESCRIPTION OF THE DRAWINGS

FIG. 1 is a diagram of the steps of the method of the invention.

FIG. 2 is a diagram of the steps of the method of the invention.

### DESCRIPTION OF THE PREFERRED EMBODIMENTS

While the invention is susceptible to various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

In the following description and in the figures, like elements are identified with like reference numerals. The use of “or” indicates a non-exclusive alternative without limitation unless otherwise noted. The use of “including” means “including, but not limited to,” unless otherwise noted.

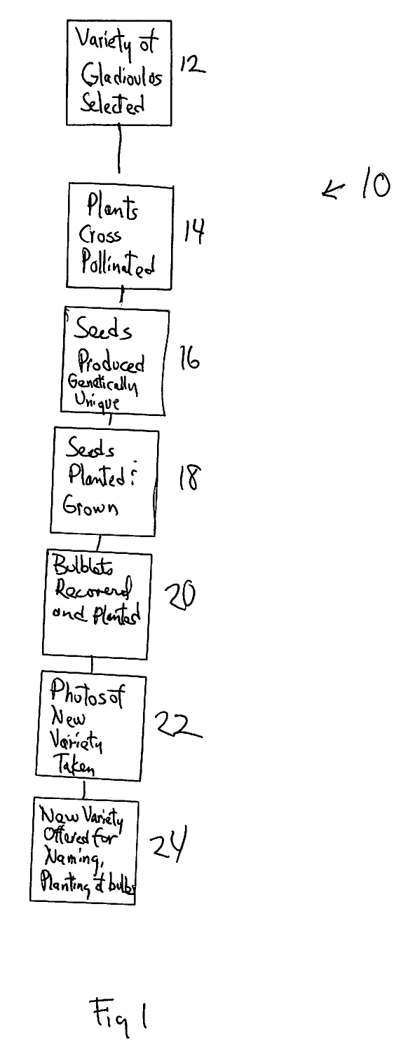

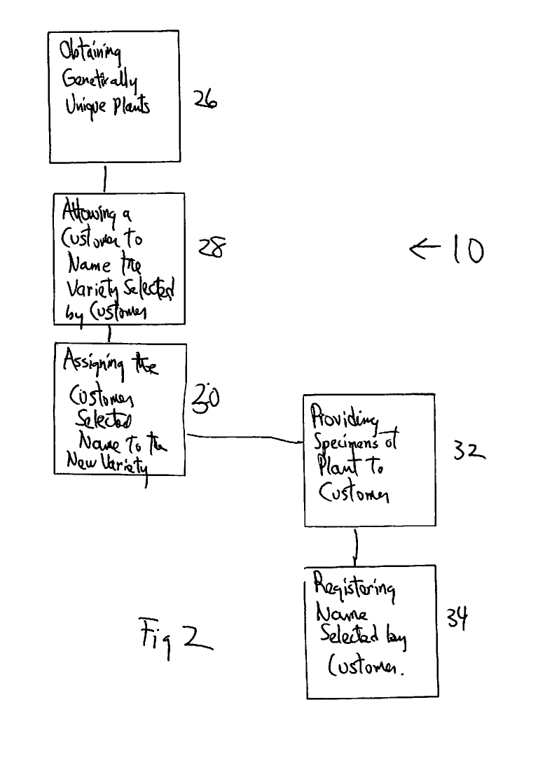

The naming and hybridization business method of the invention is further shown in the FIGS. 1 through 2.

FIG. 1 shows the naming hybridization business of the invention, which is indicated as 10. At step 12 of the process, desirable varieties of Gladiolas or other suitable plant are selected for hybridization. At step 14, the selected plants are hybridized, typically by placing the pollen of one plant onto the flower parts of another plant. At step 16, seeds from one or more of the selected specimens of plants are produced, with each seed being a genetically unique combination and different from the parent varieties. At step 18, the seeds from the selected varieties are planted and grown to maturity. When the plants have grown and flowered, they are evaluated for the desirability of their flower, based on size, color and other features. At step 20, bulblets are recovered from the desirable individuals which were grown from seed. Each of the bulblets is produced vegitatively from the plant grown from seed, and is genetically identical to the plant grown from seed. At step 22 photos of the suitable varieties of plants are taken to show a potential customer the appearance of the flowers. At step 24 the new variety is offered for sale to a customer. Typically, the sale would include a number of bulbs or bulblets which are genetically identical to the plant grown from a seed, and the customer would be offered the option of naming the new plant variety with the name of their choice. Typically the package offered for sale would include the bulbs, the naming of the flower, the entering of the selected name onto a registry of varietal names and other components of the package, which might include instructions about growing the plants and a subscription to a botanical magazine.

FIG. 2 shows another preferred embodiment of the invention. At step 26 shown in FIG. 2, genetically unique plants are obtained. The step at block 28 is the step of allowing a customer to name the variety selected by the customer. At block 28, the step of assigning the customer selected name to the new variety is shown.

The step shown in FIG. 2 can include the optional step of providing one or more living specimens of the selected plant variety to the customer to grow or give as a gift. This is shown in block 32. At block 34 is shown the optional step of registering the name selected by the customer for the new variety

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A business method which includes the steps of:
obtaining a plurality of genetically distinct living plants;
allowing a customer to select and purchase one or more plants from among the plurality of plants;
allowing said customer to select a name for the selected plant or plants; and
assigning the customer selected name to the selected plant or plants.

2. The business method of claim one which further includes the step of providing one or more specimens of the selected plant or plants to the customer for said customer to grow or give as a gift.

3. The business method of claim one which further includes the step of displaying a plurality of genetically distinct living plants for customer to review and from which to make his selection.

4. The business method of claim one which further includes the step of displaying a plurality of genetically distinct living plants for customer to review and from which to make his selection.

5. The business method of claim one which further includes a step of registering said name selected by said customer in a registry of plant variety names.

6. The business method of claim one which further includes the step of providing a pedigree of said selected plant to the customer.

7. The business method of claim 1 which includes the step of obtaining a plurality of genetically distinct Gladiolus plants by creating hybrid Gladiolus plants.

8. The business method of claim 2 which includes the step of providing a customer the option of entering certain dedication text to accompany the gift of the plant specimens.

9. The business method which includes the steps of:
obtaining a plurality of genetically distinct living plants by creating hybrid plants;
showing a display of said plurality of genetically distinct living plants;
allowing a customer to select and purchase one or more plants from among the plurality of plants;
allowing said customer to select a name for the selected plant or plants; and
assigning the customer selected name to the selected plant; and
providing one or more living specimens of the selected plant to the customer for said customer to grow or give as a gift.

10. The business method which includes the steps of:
obtaining a plurality of genetically distinct living Gladiolus plants by creating hybrid Gladiolus plants;
showing a display of said plurality of genetically distinct living plants;
allowing a customer to select and purchase one or more Gladiolus plants from among the plurality of Gladiolus plants shown;
allowing said customer to select a name for the selected Gladiolus plant or plants; and
assigning the customer selected name to the selected Gladiolus plant; and
providing one or more living Gladiolus specimens of the selected plant to the customer for said customer to grow or give as a gift; and
registering the customer selected name of the Gladiolus plant in a name and genetics registry.

11. A business method which includes the steps of:
selecting two plants of preferred varieties of genetically distinct living plants;
cross pollinating the two plants to produce seeds which are a hybrid of the two varieties;
collecting seeds produced from cross pollination of the two plants;
planting the seeds produced by cross pollination of the two plants to produce a bulb bearing mature plant;
collecting the bulbs of the mature plant, and planting them to produce mature plants genetically identical to the original mature hybrid;
offering a set of bulbs of said genetically identical plants for propagation to a customer;
and allowing a customer to name the unique variety of plant.

12. The method of claim 11 which further includes the step of registering the selected name for the variety of plant.
